(19) United States
(12) Patent Application Publication
(10) Pub. No.: US 2020/0249186 A1
(43) Pub. Date: Aug. 6, 2020

(54) SENSOR MODULE, IN PARTICULAR FOR MEASURING THE AMBIENT TEMPERATURE, THE RELATIVE HUMIDITY AND A GAS CONCENTRATION IN THE ENVIRONMENT OF THE SENSOR MODULE

(71) Applicant: SENSIRION AG, Stäfa (CH)

(72) Inventors: Dino KELLER, Stäfa (CH); Kevin MIKES, Stäfa (CH); Salomon DIETHER, Stäfa (CH); Lukas WINKLER, Stäfa (CH)

(73) Assignee: SENSIRION AG, Stäfa (CH)

(21) Appl. No.: 16/758,410

(22) PCT Filed: Oct. 23, 2018
(86) PCT No.: PCT/EP2018/079067

§ 371 Date: Apr. 23, 2020

(30) Foreign Application Priority Data
Oct. 23, 2017 (DE) 20 2017 106 413.1

(51) Int. Cl. G01N 27/04; G01N 33/00; G01N 27/22; G01K 13/00

(52) U.S. Cl. CPC G01N 27/048; G01N 33/004; G01K 13/00; G01N 27/223; G01N 33/0047

(57) ABSTRACT

The invention relates to a sensor module (1), comprising: a circuit board (2), at least one temperature sensor (3) arranged on the circuit board (2) for measuring an ambient temperature, at least one further sensor (4) arranged on the circuit board (2), which further sensor (4) generates waste heat when the further sensor (4) is operated. According to the invention, the sensor module (1) is designed for thermal decoupling of the temperature sensor (3) from the further sensor (4) and/or for dissipating the waste heat of the further sensor (4).

The present invention relates to a sensor module for measuring an ambient temperature and/or relative air humidity and in particular for measuring a concentration of a gas, e.g. CO₂ and/or volatile organic compounds, and/or of particulate matter.

The present invention is based on the problem of creating a sensor module of the afore-mentioned kind which enables a precise measurement of the ambient temperature.

This problem is solved by a sensor module having the features of claim 1 and by a device having the features of claim 32.

Preferred embodiments of these aspects of the invention are stated in the corresponding sub claims and are described below.

According to claim 1, a sensor module is disclosed, comprising:
a circuit board,
at least one temperature sensor arranged on the circuit board for measuring an ambient temperature, i.e. a temperature in an environment of the sensor module, and
at least one further sensor arranged on the circuit board, which further sensor generates waste heat when the further sensor is operated.

According to the invention, the sensor module is designed for thermal decoupling of the temperature sensor from the further sensor and/or for dissipating the waste heat of the further sensor.

A thermal decoupling is present in the sense of the present invention if a corresponding measure or a corresponding means of the sensor module reduces or ideally completely suppresses a heat effect of the respective component (e.g. further sensor and/or voltage transformer, see below) on the temperature sensor.

This has the advantage of counteracting a falsification of the measured ambient temperature by the waste heat generated by the sensor module, which increases the accuracy of the sensor module with regard to temperature measurement.

According to an embodiment of the sensor module according to the invention, for thermal decoupling of the temperature sensor from the further sensor, the temperature sensor comprises a minimum distance to the further sensor, wherein particularly the minimum distance is larger than 1.5 cm, in particular larger than 2 cm.

According to a further embodiment, said minimum distance is at least 60% of a maximum width of the circuit board.

Furthermore, according to an embodiment, the temperature sensor is arranged on the circuit board diagonally and/or offset to the further sensor, in particular in order to further maximize a distance between the two sensors.

Furthermore, according to an embodiment of the sensor module, the circuit board comprises at least one slot which is arranged between the temperature sensor and the further sensor for thermal decoupling of the temperature sensor from the further sensor. Preferably, according to an embodiment, the circuit board comprises at least two slots, preferably at least three slots, preferably four slots.

In particular, the circuit board may comprise one or more or any combination of the following slots:
a (in particular first) slot, which is arranged in particular between the temperature sensor and the further sensor (e.g. in the second section, in particular at the transition between the second and the third section of the circuit board, see below);
a further (in particular second) slot, which is arranged in particular between the temperature sensor and the further sensor (e.g. in the second section, in particular at the transition between the first and the second section of the circuit board, see below); this slot serves in particular as a blockade to concentrate heat at the location of the first section of the circuit board,
a further (in particular third) slot, which is formed in particular in the third section of the circuit board, wherein this slot is arranged in particular between the temperature sensor and the further sensor, and wherein this slot extends (in particular openly annular) around or encompasses a circuit board region of the third section of the circuit board, for example, wherein the temperature sensor is arranged on this circuit board region; this slot particularly causes a low thermal mass of said (peninsular) circuit board region, so that rapid response times to temperature changes are achieved, i.e., the smaller the thermal mass of this circuit board region, the faster it assumes the ambient temperature when the latter changes rapidly. Since the temperature sensor also assumes the temperature of this circuit board region, the temperature sensor can follow changes in the ambient temperature as quickly as possible;
a further (in particular fourth) slot formed in particular in the second section of the circuit board, this slot being formed in the circuit board, e.g. underneath the connector part; this slot also serves as a blockage which promotes heat dissipation via the connector part.

According to a further embodiment of the sensor module according to the invention, the circuit board comprises a first section (in particular in the form of a first end section of the circuit board), a second section (in particular in the form of a central section of the circuit board) and a third section (in particular in the form of a second end section of the circuit board), wherein the first and third sections are connected to one another via the second section of the circuit board.

Here, according to an embodiment of the invention, the further sensor is arranged on the first section of the circuit board, and/or the temperature sensor is arranged on the third section of the circuit board.

According to a further embodiment of the invention, the first slot is formed in the second section of the circuit board. The second or central section thus separates the sections of the circuit board, which comprise the temperature sensor and the further sensor, from each other so that waste heat of the further sensor is generated as far away as possible from the temperature sensor, wherein furthermore the first slot of the circuit board reduces heat transfer or heat conduction from the first section to the third section.

In particular, it is provided that the first slot is arranged at a transition from the second to the third section of the circuit board.

Furthermore, according to an embodiment, the second slot is formed in the second section of the circuit board, wherein the second slot is arranged in particular at a transition from the second to the first section.

Furthermore, according to an embodiment of the sensor module according to the invention, the temperature sensor is electrically conductively connected to at least one conductive path of the circuit board extending along the circuit board, wherein the at least one conductive path is particularly configured in such a way that heat transfer to the temperature sensor is as low as possible.

In particular, it is furthermore provided according to an embodiment that the at least one conductive path extends from the first section of the circuit board to the third section of the circuit board.

Furthermore, according to an embodiment, the at least one conductive path preferably extends around the first slot so that the first slot is arranged between two opposing sections of the at least one conductive path.

Furthermore, according to an embodiment of the invention, the at least one conductive path extends past the second slot.

Furthermore, according to an embodiment of the invention, the third slot extends around a circuit board region of the third section of the circuit board, on which circuit board region the temperature sensor is arranged. Thus, said circuit board region is connected to the rest of the circuit board only by a bar extending between two ends of the third slot. The at least one conductive path extends to the temperature sensor via this bar, wherein the bar is arranged on a side of the temperature sensor which faces away from the first section of the circuit board.

Furthermore, according to an embodiment of the sensor module according to the invention, the sensor module further comprises a voltage transformer for supplying the temperature sensor and the further sensor with an operating voltage, wherein the sensor module is designed for thermal decoupling of the temperature sensor from the voltage transformer.

Here, too, it is preferably provided in accordance with an embodiment that the voltage transformer is arranged adjacent to the further sensor on the first section of the circuit board.

Here, too, in accordance with an embodiment, the temperature sensor comprises a minimum distance from the voltage transformer for thermal decoupling of the temperature sensor from the voltage transformer, wherein in particular the minimum distance is larger than 1.5 cm, in particular larger than 2 cm.

Furthermore, according to an embodiment, the first and/or the second and/or the third slot (see above) is/are also arranged between the temperature sensor and the voltage transformer, in particular in order to reduce heat transfer from the voltage transformer to the temperature sensor.

According to another embodiment, the sensor module comprises a plug connector part for electrically contacting the sensor module, which plug connector part is arranged on the circuit board. The plug connector part is preferably designed to make a plug connection with a further plug connector part. The plug connector part can be designed as a socket, for example, and the further plug connector part can be designed as a plug which can be connected to the plug connector part in a form-fitting and/or force-fitting manner, e.g. by plugging it into the plug connector part.

Furthermore, according to an embodiment, the plug connector part is arranged on the circuit board between the temperature sensor and the further sensor and/or the voltage transformer for thermal decoupling of the temperature sensor from the further sensor. In particular, the plug connector part forms a shield that keeps waste heat (especially heat radiation) of the additional sensor or of the voltage transformer away from the temperature sensor.

According to a further embodiment of the invention, the plug connector part is arranged on the second section of the circuit board.

Furthermore, according to an embodiment of the invention, the fourth slot is arranged below the connector part, i.e. the connector part covers the fourth slot.

Furthermore, according to an embodiment of the invention, the circuit board of the sensor module comprises a plurality of metallic conductive paths for connecting the electrical components of the sensor module (in particular temperature sensor, further sensor, voltage transformer and/or plug connector part), wherein a density of the conductive paths is larger in the first section than in the second and third sections of the circuit board, so that in particular the waste heat generated by the further sensor and/or the voltage transformer can be concentrated on the first section of the circuit board.

Furthermore, according to an embodiment, for dissipating the waste heat of the first section of the circuit board, the first section of the circuit board comprises at least one metallic cooling element which is provided between adjacent conductive paths of the first section of the circuit board. This at least one metallic cooling element, which can be designed as a flat metallization of the circuit board, comprises in particular a sufficient distance from adjacent conductive paths so that no short circuit can occur. The at least one cooling element can extend in the plane of the conductive paths.

Furthermore, according to an embodiment of the invention, this at least one metallic cooling element comprises a region which extends along an edge of the first section of the circuit board, wherein this region is exposed at least in sections, i.e. contacting the ambient atmosphere, according to an embodiment of the sensor module according to the invention in order to increase the radiation or emission of the waste heat. The at least one cooling element, on the other hand, can be insulated from the environment, like the conductive paths.

Furthermore, according to a preferred embodiment of the sensor module, waste heat generated in the first section of the circuit board by the further sensor and/or the voltage transformer can be dissipated via the plug connector part (which is arranged in particular on the adjacent second section of the circuit board), in particular via the further plug connector part and a cable connected thereto, if the further plug connector part is connected to the plug connector part.

According to a further embodiment of the sensor module according to the invention, the temperature sensor is connected to a flat metal element, in particular a flat metallization, for good thermal coupling, which is arranged on a lower side of the circuit board.

In this regard the lower side (or unpopulated side) of the circuit board faces away from an upper side (or a populated side) of the circuit board, wherein the temperature sensor and/or the further sensor and/or the voltage transformer and/or the plug connector part are arranged on the upper side.

Furthermore, the temperature sensor may also be designed to measure the relative humidity (RH) according to an embodiment of the invention, e.g. according to DE20201105119.

According to a further embodiment of the invention, the further sensor can be a gas sensor and/or a particulate matter sensor.

In particular, the further sensor can be designed as a particulate matter sensor designed to measure a particulate matter concentration in an environment of the sensor module, e.g. according to WO2017054098.

Alternatively, the additional sensor can be a gas sensor designed to measure a gas concentration in an environment of the sensor module.

In particular, this gas sensor can be a MOX gas sensor, e.g. according to EP2765410, or an optical gas sensor, e.g. according to EP3144663, or an electrochemical gas sensor, e.g. according to EP2896962.

The gas sensor is especially designed to measure a CO₂ concentration and/or a concentration of at least one volatile organic compound.

The gas sensor may have a gas sensor chip or may be designed as a gas sensor chip. The gas sensor chip may contain a semiconductor substrate, for example out of silicon, in which semiconductor substrate a circuit may be integrated. Different layers, for example CMOS layers, can be used to create the circuit.

The gas sensor chip preferably comprises a front and a back side, wherein the sensitive layer is preferably located on the front side. The sensitive layer can be located on the semiconductor substrate or on a layer, for example on one of the CMOS layers, which belong to the semiconductor substrate. If a circuit is included in the same gas sensor chip, the sensitive layer may be connected to it so that signals from the sensitive layer can be pre-processed in the circuit. The integrated circuit can also be used, for example, to control a heater (see below).

The sensitive layer can be made out of a material that is sensitive to one or more analytes. The sensitive layer can contain several individual layer regions, which are arranged next to each other and separated from each other to form a sensor array, containing a group of sensor cells, where a sensor cell is understood as a functional unit of the gas sensor, which can be read out individually. Preferably, in the embodiment of the sensor arrangement, each or at least some of the layer regions is suitable for detecting analytes and in particular for detecting different analytes. The analytes may include without limitation one or more of the following, for example CO₂, NOX, ethanol, CO, ozone, ammonia, formaldehyde, H₂O or xylene.

In particular, the sensitive layer can contain a metal-oxide material, especially a semiconductor metal-oxide material, and in particular metal-oxide materials with a different composition per layer region. A metal oxide material can generally contain one or more of: tin oxide, zinc oxide, titanium oxide, tungsten oxide, indium oxide or gallium oxide. Such metal oxides can be used to detect analytes such as volatile hydrocarbons (VOCs), carbon monoxide, nitrogen dioxide, methane, ammonia, or hydrogen sulphide.

Metal-oxide sensors are based on the concept that at elevated temperatures of the sensitive metal-oxide layer in the range of more than 100° C., especially between 250° C. and 350° C., gaseous analytes interact with the metal-oxide layer. As a result of this catalytic reaction, the conductivity of the sensitive layer can change and this change can be measured. Consequently, such chemical sensors are also called high-temperature chemoresistors, because at high temperatures of the sensitive layer a chemical property of the analyte is converted into an electrical resistance. Preferably, a gas can be examined by means of such a gas sensor, at least for the presence or absence of the analyte or analytes to which the sensor is sensitive.

In another embodiment, the sensitive layer can contain a polymer which in an embodiment can be sensitive to H₂O, so that the sensor can be a humidity sensor. By measuring a capacitance or resistance of such a polymer layer, information about the gas interacting with the sensitive layer can be obtained.

According to another embodiment, the gas sensor comprises a gas sensor chip with a sensitive layer sensitive to a gas or to components of the latter, and a heater.

A further aspect of the present invention relates to a device comprising a sensor module according to the invention, wherein the sensor module is arranged in such a way that, with respect to an air flow flowing in the device, the temperature sensor is arranged upstream of the further sensor and/or of the voltage transformer, or such that the air flow flows over the circuit board perpendicular to a circuit board axis, wherein the first and the third section face each other in the direction of the circuit board axis.

Further features, advantages and embodiments of the present invention shall be explained in the following on the basis of the figures, wherein:

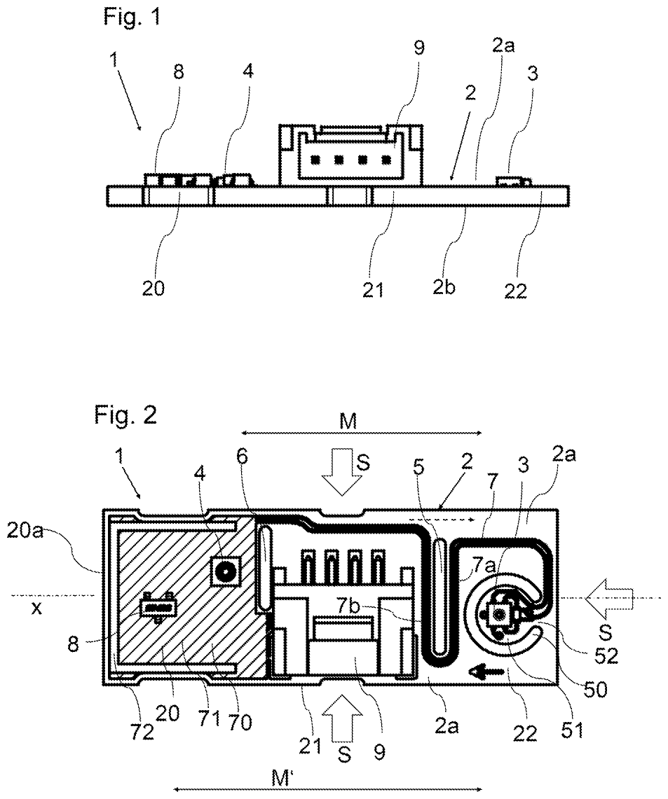

FIG. 1 shows a side view of a sensor module according to the invention;

FIG. 2 shows a top view of an upper side of the sensor module shown in FIG. 1;

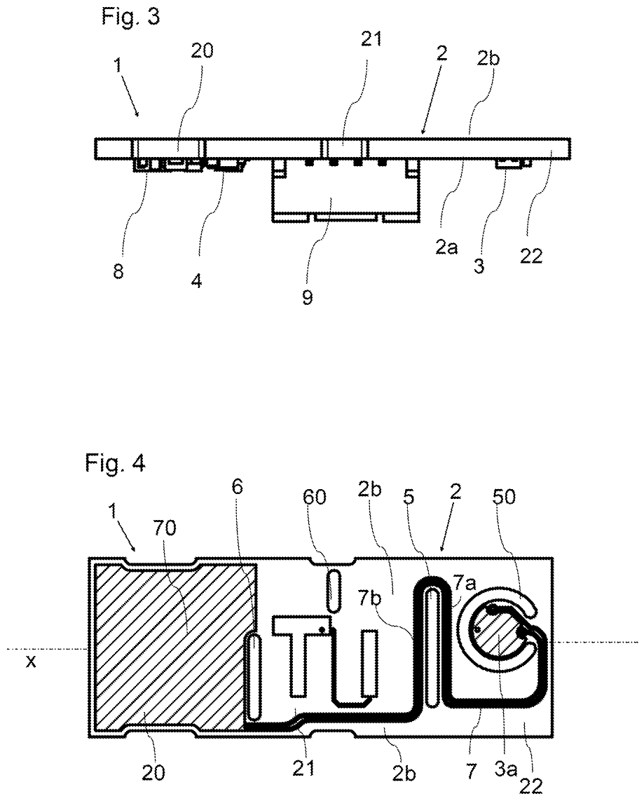

FIG. 3 shows another side view of the sensor module shown in FIGS. 1 and 2; and

FIG. 4 shows a top view of a bottom side of the sensor module shown in FIGS. 1 to 3.

FIGS. 1 to 4 show a sensor module 1 according to the invention. The sensor module 1 comprises a circuit board 2, at least one temperature sensor 3 arranged on the circuit board 2 for measuring an ambient temperature and at least one further sensor 4 arranged on the circuit board 2, which generates waste heat when the further sensor 4 is operated. The further sensor 4 is, for example, a gas sensor which is particularly designed to measure the concentration of at least one gas in the atmosphere surrounding the sensor module. The two sensors 3, 4 (and in particular the voltage transformer 8 and the plug connector part 9, see below) are preferably arranged on an upper side 2a of the circuit board 2, which faces away from a lower side 2b of the circuit board 2. The upper side 2a is also called the populated side; the lower side 2b can represent a non-populated side of the circuit board 2.

Since further sensors 4, such as a gas sensor, usually generate a relatively large amount of waste heat which could falsify the measurement result of the temperature sensor 3, it is provided according to the invention that the sensor module 1 is designed for thermal decoupling of the temperature sensor 3 from the further sensor or gas sensor 4 and/or for dissipating the waste heat of the further sensor 4.

Furthermore, the sensor module 1 can also comprise a voltage transformer 8 which also produces waste heat and which serves, for example, to provide an operating voltage for the temperature sensor 3 and/or for the additional sensor 4. The sensor module 1 is then preferably also designed for thermal decoupling of temperature sensor 3 from voltage transformer 8 or for dissipation of the waste heat of voltage transformer 8.

In order to achieve or improve said thermal decoupling, it can be provided that the temperature sensor 3 comprises a minimum distance M to the further sensor 4 or a minimum distance M′ to the voltage transformer 8.

As shown in FIGS. 1 to 4, the circuit board 2 can also comprise a first, a second and a third section 20, 21, 22, wherein the first section 20 or end section 20 is connected to the third section 22 or end section 22 via the second section 21.

For thermal decoupling of the temperature sensor 3 from the further sensor 4 or, if necessary, from the voltage transformer 8, it can also be provided that the circuit board 2 comprises, for example, a first slot 5, which is arranged between the temperature sensor 3 and the further sensor 4, in particular in the second section 21 of the circuit board, preferably at the transition from the second section 21 to the third section 22 of the circuit board 2. The circuit board 2 can also comprise a second slot 6. The two slots 5, 6 can be arranged parallel to each other, for example. Through such slots 5, 6, heat conduction is reduced from the first section 20 of the circuit board 2, on which the further sensor 4 and the voltage transformer 8 are arranged, to the third section 22 of the circuit board 2, on which the temperature sensor 3 to be protected against waste heat is located.

Furthermore, the temperature sensor 3 can be electrically conductively connected to at least one conductive path 7 of the circuit board 2, the at least one conductive path 7 being configured in such a way that it transports as little waste heat as possible from the further sensor 4 or the voltage transformer 8 to the temperature sensor 3. The at least one conductive path 7 can extend from the first section 20 of the circuit board 2 to the third section 22 of the circuit board 2. Furthermore, the at least one conductive path 7 can pass the second slot 6 and be routed around the first slot 5 so that the first slot 5 is located between two opposing sections 7a, 7b of the at least one conductive path 7 (see FIGS. 2 and 4). The relatively long length of at least one conductive path 7 allows waste heat generated in the first section 20 of the circuit board 2 to be easily dissipated during transport via the said conductive path 7 on its way to the temperature sensor 3. In particular, with regard to the first slot 5, there are two possible paths for the heat flow. This can be done, for example, around the first slot 5 via conductive path 7 or via the circuit board 2 along an edge of the circuit board 2 (dashed arrow in FIG. 2). The metal of conductive path 7 conducts heat better than the circuit board 2 itself, but the length and cross-section of conductive path 7 and the dimensions of the first slot 5 are chosen such that neither of the two possible paths of heat flow dominates strongly.

Furthermore, the circuit board 2 can comprise a third slot 50 in the third section 22, which extends around a circuit board region 51 of the third section 22 of the circuit board 2 (e.g. open ring-shaped), wherein the temperature sensor 3 is arranged on this circuit board region 51 (on the upper side 2a of the circuit board 2), whereas a flat metal element 3a of the temperature sensor 3 is arranged on this circuit board region 51 for rapid thermal coupling on the lower side 2b of the circuit board 2. Due to the third slot 50, said circuit board region 51 is connected to the rest of the circuit board 2 only via a bar 52, wherein this bar 52 extends between two ends of the third slot 50. The at least one conductive path 7 extends via this bar 52 to the temperature sensor 3, wherein the bar 52 is arranged on a side of the temperature sensor 3 which faces away from the first section 20 of the circuit board 2. Furthermore, the sensor module 1 can comprise a plug connector part 9 for electrically contacting the sensor module 1, which is arranged on the circuit board 2 preferably in the second or central section 21 of the circuit board, e.g. between the two slots 5, 6. The plug connector part 9 can be designed to make a plug connection with a further plug connector part (not shown) which can be provided e.g. at one end of a cable, so that an electrical connection can be established between the cable and the sensor module 1. Below the plug connector part 9, the circuit board 2 can comprise a fourth slot 60 in the second section 21 of the circuit board 2, which causes waste heat to be emitted via the plug connector part 9.

Due to the arrangement of the plug connector part 9 in the second section 21 of the circuit board 2 between the further sensor 4 and the voltage transformer 8 on the one hand and the temperature sensor 3 on the other hand, the plug connector part 9 forms a shield which shields the temperature sensor 3 from the waste heat (especially heat radiation) of the further sensor 4 and the voltage transformer 8, which are arranged on the first section 20 of the circuit board 2. Furthermore, waste heat from the first section 20 of the circuit board 2 can be dissipated via the plug connector part 9 (e.g. into a cable connected to the connector part 9).

Furthermore, the conductive paths 7, 70 of the sensor module 1 can have a maximum density in the first section 20 of the circuit board 2 (see particularly FIG. 4), so that in the first section 20 of the circuit board 2, waste heat generated by the further sensor 4 and/or the voltage transformer 8 can be concentrated there at first.

To dissipate waste heat from the first section 20 of the circuit board 2, the first section 20 can comprise at least one metallic cooling element 71, which is arranged between adjacent conductive paths 70 or next to a conductive path 70 of the first section 21 of the circuit board 2. The at least one metallic cooling element 71 can comprise a region 72 which extends along an edge 20a of the first section 21 of the circuit board 2, wherein this region 72 is at least partially exposed to increase the emission of waste heat.

According to a further aspect of the invention, a device is disclosed which comprises a sensor module 1 according to the invention, wherein this sensor module (cf. FIG. 2) is arranged in such a way that, with respect to an air flow S flowing in the device, the temperature sensor 3 is arranged upstream of the further sensor 4 and/or of the voltage transformer 8. Alternatively, the air flow S can flow across the circuit board 2 perpendicular to a circuit board axis x, wherein the first and third sections 20, 22 of the circuit board 2 face each other in the direction of the circuit board axis x.

1. A sensor module (1), comprising:
a circuit board (2),
at least one temperature sensor (3) arranged on the circuit board (2) for measuring an ambient temperature
at least one further sensor (4) arranged on the circuit board (2), which further sensor (4) generates waste heat when the further sensor (4) is operated,
characterized in that
the sensor module (1) is designed for thermal decoupling of the temperature sensor (3) from the further sensor (4) and/or for dissipating the waste heat of the further sensor (4).

2. The sensor module according to claim 1, characterized in that for thermal decoupling of the temperature sensor (3) from the further sensor (4) the temperature sensor (3) comprises a minimum distance (M) from the further sensor (4), wherein in particular the minimum distance is larger than 1.5 cm, in particular larger than 2 cm; and/or in that said minimum distance (M) is at least 60% of a maximum width of the circuit board (2).

3. The sensor module according to one of the preceding claims, characterized in that the temperature sensor (3) is arranged on the circuit board (2) diagonally and/or offset relative to the further sensor (4), in particular in order to further maximize a distance between the two sensors (3, 4).

4. The sensor module according to one of the preceding claims, characterized in that for thermal decoupling of the temperature sensor (3) from the further sensor (4) the circuit board (2) comprises one or more of the following slots:
a first slot (5), which is arranged in particular between the temperature sensor (3) and the further sensor (4),
a second slot (6), which is arranged in particular between the temperature sensor (3) and the further sensor (4),
a third slot (50), which is arranged in particular between the temperature sensor (3) and the further sensor (4),
a fourth slot (60).

5. The sensor module according to one of the preceding claims, characterized in that the circuit board (2) comprises a first section (20), a second section (21) and a third section (22), wherein the first and third sections (20, 22) are connected to each other via the second section (21).

6. The sensor module according to claim 5, characterized in that the further sensor (4) is arranged on the first section (20) of the circuit board (2) and/or that the temperature sensor (3) is arranged on the third section (22) of the circuit board (2).

7. The sensor module according to claims 4 and 6, characterized in that the first slot (5) is formed in the second section (21) of the circuit board (2), wherein the first slot (5) is arranged in particular at a transition to the third section (22).

8. The sensor module according to claim 4 and according to one of claims 5 to 7, characterized in that the second slot (6) is formed in the second section (21) of the circuit board (2), wherein the second slot (6) is arranged in particular at a transition to the first section (20).

9. The sensor module according to one of the preceding claims, characterized in that the temperature sensor (3) is electrically conductively connected to at least one conductive path (7) of the circuit board (2).

10. The sensor module according to claims 5 and 9, characterized in that the at least one conductive path (7) extends from the first section (20) of the circuit board (2) to the third section (22) of the circuit board (2).

11. The sensor module according to claim 4 and according to claim 9 or 10, characterized in that the at least one conductive path (7) extends around the first slot (5) so that the first slot (5) is arranged between two opposing sections (7a, 7b) of the at least one conductive path (7).

12. The sensor module according to claim 4 and according to one of claims 9 to 11, characterized in that the at least one conductive path (7) extends past the second slot (6).

13. The sensor module according to claim 4 or according to one of claims 5 to 12 if referring to claim 4, characterized in that the third slot (50) extends around a circuit board region (51) of the third section (22) of the circuit board (2), wherein the temperature sensor (3) is arranged on this circuit board region (51).

14. The sensor module according to one of the preceding claims, characterized in that the sensor module (1) further comprises a voltage transformer (8) for supplying the temperature sensor (3) and/or the further sensor (4) with an operating voltage, wherein the sensor module (1) is designed for thermal decoupling of the temperature sensor (3) from the voltage transformer (8).

15. The sensor module according to claims 5 and 14, characterized in that the voltage transformer (8) is arranged adjacent to the further sensor (4) on the first section (20) of the circuit board (2).

16. The sensor module according to claim 14 or 15, characterized in that for thermal decoupling of the temperature sensor (3) from the voltage transformer (8), the temperature sensor (3) comprises a minimum distance (M′) from the voltage transformer (8), wherein in particular the minimum distance (M′) is larger than 1.5 cm, in particular larger than 2 cm.

17. The sensor module according to claim 4 or 7 and according to one of claims 14 to 16, characterized in that the first slot (5) is also arranged between the temperature sensor (3) and the voltage transformer (8).

18. The sensor module according to one of the preceding claims, characterized in that the sensor module (1) comprises a plug connector part (9) for electrically contacting the sensor module (1), which plug connector part (9) is arranged on the circuit board (2).

19. The sensor module according to claim 18, characterized in that for thermal decoupling of the temperature sensor (3) from the further sensor (4), the plug connector part (9) is arranged between the temperature sensor (3) and the further sensor (4) on the circuit board (2).

20. The sensor module according to claim 14 and according to claim 18 or 19, characterized in that for thermal decoupling of the temperature sensor (3) from the voltage transformer (8), the plug connector part (9) is arranged between the temperature sensor (3) and the voltage transformer (8).

21. The sensor module according to claim 5 and according to one of claims 18 to 20, characterized in that the plug connector part (9) is arranged on the second section (21) of the circuit board (2).

22. The sensor module according to claim 4 and according to one of claims 18 to 21, characterized in that the fourth slot (60) is arranged under the plug connector part (9).

23. The sensor module according to claim 5 or according to one of claims 6 to 22 if referring to claim 5, characterized in that the circuit board (2) of the sensor module (1) comprises a plurality of conductive paths (7, 70), wherein a density of the conductive paths is larger in the first section (20) of the circuit board (2) than in the second and third sections (21, 22) of the circuit board (2).

24. The sensor module according to claim 5 and according to claim 18 or one of claims 19 to 23 is referring to claim 18, characterized in that waste heat generated in the first section (20) of the circuit board (2) can be dissipated via the plug connector part (9).

25. The sensor module according to claim 23 or according to one of the claims 19 to 23 if referring to claim 24, characterized in that for dissipating waste heat of the first section (20) of the circuit board (2), the circuit board (2) comprises in the first section (20) at least one metallic cooling element (71) which is arranged between adjacent conductive paths (70) of the first section (20) of the circuit board (2).

26. The sensor module according to claim 25, characterized in that the at least one metallic cooling element (71) comprises a region (72) which extends along an edge (20a) of the first section (20) of the circuit board (2), wherein this region (72) is exposed at least in sections to increase the emission of waste heat.

27. The sensor module according to one of the preceding claims, characterized in that the temperature sensor (3) is connected to a flat metal element (3a) for thermal coupling, which flat metal element (3a) is arranged on a lower side (2b) of the circuit board (2).

28. The sensor module according to one of the preceding claims, characterized in that the temperature sensor (3) is also designed to measure the relative humidity of air.

29. The sensor module according to one of the preceding claims, characterized in that the further sensor (4) is an environmental sensor.

30. The sensor module according to one of the preceding claims, characterized in that the further sensor (4) is a particulate matter sensor which is designed to measure a particulate matter concentration in an environment of the sensor module (1).

31. The sensor module according to one of the preceding claims, characterized in that the further sensor (4) is a gas sensor which is designed to measure a gas concentration in an environment of the sensor module (1).

32. A Device comprising a sensor module (1) according to one of the preceding claims, characterized in that the sensor module (1) is arranged in such a way that, with respect to an air flow (S) flowing in the device, the temperature sensor (3) is arranged upstream of the further sensor (4) and/or of the voltage transformer (8); or in that the air flow (S) flows over the circuit board (2) perpendicular to a circuit board axis (x), wherein the first and third sections (20, 22) of the circuit board (2) face each other in the direction of the circuit board axis (x).
